SEQUENCE LISTING (18 sequences)

SEQ ID NO: 1 - DNA, 38 bp, synthetic construct
  source 1..38: mol_type = other DNA
tgtaaaacgacggccagtagtggttctcgcttacaccg

SEQ ID NO: 2 - DNA, 20 bp, synthetic construct
  source 1..20: mol_type = other DNA
aggtataggaggcgggtgtt

SEQ ID NO: 3 - DNA, 271 bp, synthetic construct
  source 1..271: mol_type = other DNA
agtggttctcgcttacaccgaacctgattctgaatactctgcagacgcagatgaagaaatgacacctgaggcgattatccacgctcgtcaggttgttgggcaacaagggcgtcttggttactttgacctacatccagaacgaagacctgccgctgtcaatcaaaatgatgaagaccctagattgcgccttgccagagacgacagtgatgatgatgatgatgatgatgaacaagtatatggtcgctccgagcaacacccgcctcctatacct

SEQ ID NO: 4 - DNA, 274 bp, synthetic construct
  source 1..274: mol_type = other DNA
agtggttctcgcttacaccgaacctgattctgaatactctgcagacgcagatgaagaaatgacacctgaggcgattatccacgctcgtcaggttgttgggcaacaagggcgtcttggttactttgacctacatccagaacgaagacctgccgctgtcaatcaaaatgatgaagaccctagattgcgccttgccagagacgacagtgatgatgatgatgatgatgatgatgaacaagtatatggtcgctccgagcaacacccgcctcctatacct

SEQ ID NO: 5 - DNA, 38 bp, synthetic construct
  source 1..38: mol_type = other DNA
tgtaaaacgacggccagtacagggctcattgtaccgtg

SEQ ID NO: 6 - DNA, 20 bp, synthetic construct
  source 1..20: mol_type = other DNA
ggctcctagcgctattcgtt

SEQ ID NO: 7 - DNA, 235 bp, synthetic construct
  source 1..235: mol_type = other DNA
acagggctcattgtaccgtgctcagacgggcaccctattcaaacgcatttggagaacacccgaaatggcgatgctgtttggtggggatgcggtgagctttgcatgggtttgcaggaagaggatgagtcggatttagaggatgcagatgaacggactttgctacaacaacaacaacaacaacaacatgggcatgcttcatcaccattgcccaatcgaacgaatagcgctaggagcc

SEQ ID NO: 8 - DNA, 238 bp, synthetic construct
  source 1..238: mol_type = other DNA
acagggctcattgtaccgtgctcagacgggcaccctattcaaacgcatttggagaacacccgaaatggcgatgctgtttggtggggatgcggtgagctttgcatgggtttgcaggaagaggatgagtcggatttagaggatgcagatgaacggactttgctacaacaacaacaacaacaacaacaacatgggcatgcttcatcaccattgcccaatcgaacgaatagcgctaggagcc

SEQ ID NO: 9 - DNA, 241 bp, synthetic construct
  source 1..241: mol_type = other DNA
acagggctcattgtaccgtgctcagacgggcaccctattcaaacgcatttggagaacacccgaaatggcgatgctgtttggtggggatgcggtgagctttgcatgggtttgcaggaagaggatgagtcggatttagaggatgcagatgaacggactttgctacaacaacaacaacaacaacaacaacaacatgggcatgcttcatcaccattgcccaatcgaacgaatagcgctaggagcc

SEQ ID NO: 10 - DNA, 38 bp, synthetic construct
  source 1..38: mol_type = other DNA
tgtaaaacgacggccagtacaacacatgcaccaatggc

SEQ ID NO: 11 - DNA, 20 bp, synthetic construct
  source 1..20: mol_type = other DNA
tgcctggttgatgaagctga

SEQ ID NO: 12 - DNA, 236 bp, synthetic construct
  source 1..236: mol_type = other DNA
acaacacatgcaccaatggccatcaaataataataaaataactattcattcagccttagggaacagcgtacgagtcggacatttacaatagtgtgtgtgtgtactgcattcaaccaaactttggactgtaacgtgtacttcactctgccaacatgatcatgaggtgaagcatgttttcaagtcactcaccacactctctcacacacatgatggctttcagcttcatcaaccaggca

SEQ ID NO: 13 - DNA, 238 bp, synthetic construct
  source 1..238: mol_type = other DNA
acaacacatgcaccaatggccatcaaataataataaaataactattcattcagccttagggaacagcgtacgagtcggacatttacaatagtgtgtgtgtgtgtactgcattcaaccaaactttggactgtaacgtgtacttcactctgccaacatgatcatgaggtgaagcatgttttcaagtcactcaccacactctctcacacacatgatggctttcagcttcatcaaccaggca

SEQ ID NO: 14 - DNA, 240 bp, synthetic construct
  source 1..240: mol_type = other DNA
acaacacatgcaccaatggccatcaaataataataaaataactattcattcagccttagggaacagcgtacgagtcggacatttacaatagtgtgtgtgtgtgtgtactgcattcaaccaaactttggactgtaacgtgtacttcactctgccaacatgatcatgaggtgaagcatgttttcaagtcactcaccacactctctcacacacatgatggctttcagcttcatcaaccaggca

SEQ ID NO: 15 - DNA, 38 bp, synthetic construct
  source 1..38: mol_type = other DNA
tgtaaaacgacggccagtgcccgtttgatgcaacatgt

SEQ ID NO: 16 - DNA, 20 bp, synthetic construct
  source 1..20: mol_type = other DNA
atctccgtgatcatgcctgc

SEQ ID NO: 17 - DNA, 267 bp, synthetic construct
  source 1..267: mol_type = other DNA
gcccgtttgatgcaacatgttgattgacgactagattggacctgcaatgcaaactaataataataataataataataataaagtcagccggcgtgtagaaatctgacacttacagtaagagtgcagatcacccaagtaggttatgagaagtatatatatacactttatacgattacgtgtaacatcacatgatatcactttttactttctacgtgacatttcccactccaaagtccaatacttccttgcaggcatgatcacggagat

SEQ ID NO: 18 - DNA, 270 bp, synthetic construct
  source 1..270: mol_type = other DNA
gcccgtttgatgcaacatgttgattgacgactagattggacctgcaatgcaaactaataataataataataataataataataaagtcagccggcgtgtagaaatctgacacttacagtaagagtgcagatcacccaagtaggttatgagaagtatatatatacactttatacgattacgtgtaacatcacatgatatcactttttactttctacgtgacatttcccactccaaagtccaatacttccttgcaggcatgatcacggagat

(19) United States
(12) Patent Application Publication
(10) Pub. No.: US 2024/0218464 A1
(43) Pub. Date: Jul. 4, 2024

(54) DNA BARCODE FOR SCREENING FLOCCULARIA LUTEOVIRENS WITH HIGH TOTAL FAT CONTENT

(71) Applicant: XIZANG VOCATIONAL TECHNICAL COLLEGE, LHASA (CN)

(72) Inventors: MANJUN YANG, LHASA (CN); CAIXIA ZHANG, LHASA (CN); YISHUI CAI, LHASA (CN)

(21) Appl. No.: 18/418,417

(22) Filed: Jan. 22, 2024

Related U.S. Application Data
(63) Continuation of application No. PCT/CN2022/109892, filed on Aug. 3, 2022.

(30) Foreign Application Priority Data
Nov. 19, 2021 (CN) 202111401641.4

(51) Int. Cl. C12Q 1/6895; C12Q 1/6806; C12Q 1/6858; C12Q 1/686; G01N 27/447

(52) U.S. Cl. CPC C12Q 1/6895; C12Q 1/6806; C12Q 1/6858; C12Q 1/686; G01N 27/44726; C12Q 2600/156

(57) ABSTRACT

The present invention discloses a DNA barcode for screening Floccularia luteovirens with high total fat content, belonging to the technical field of screening of edible fungi germplasm resources. Compared with a traditional breeding method and other existing DNA barcode technologies, the present invention has the advantages of time saving, labor saving, money saving, accuracy and high efficiency, plays a positive role in genetic breeding of high-quality Floccularia luteovirens, and also provides an effective method for identification and protection of germplasm resources.

### SEQUENCE LISTING

The instant application contains a Sequence Listing which has been submitted electronically in ST.26 format and is hereby incorporated by reference in its entirety. Said ST.26 copy, created on Jan. 19, 2024, is named Sequence Listing.xml and is 19,311 bytes in size.

### TECHNICAL FIELD

The present invention relates to the technical field of screening of edible fungi germplasm resources, in particular to a DNA barcode for screening Floccularia luteovirens with a high total fat content.

### BACKGROUND

Floccularia luteovirens in a gold yellow color is also known as yellow mushroom and golden mushroom. Floccularia luteovirens is mainly distributed in Qinghai-Tibet Plateau; and the main producing areas include Dangxiong County of Tibet Autonomous Region, Qilian County of Qinghai Province and Shiqu County of Sichuan Province, and the quality of these three main producing areas is the best. Floccularia luteovirens is a high-quality edible fungus with a unique flavor, which cannot be cultivated artificially at present. Main indexes to evaluate the nutritional value, flavor and biological activity of Floccularia luteovirens include: high contents and strong antioxidant activity of total soluble proteins, total soluble amino acids, total polyphenols, total polysaccharides and total fat. It is very difficult to screen high-quality strains by traditional methods. In addition, due to high altitude of main producing areas of the distribution, it is also difficult to collect samples. In order to realize development and utilization of Floccularia luteovirens, it is especially important and urgent to screen high-quality Floccularia luteovirens strains through assistance of a DNA barcode molecular identification technology. In different producing places, Floccularia luteovirens has different nutritional values, different flavors, different biological activities and different market prices. In the past, the breeding of Floccularia luteovirens was mainly determined by a morphological method combined with beneficial content indexes.

However, due to the influence of the special climate environment of Qinghai-Tibet Plateau, the phenomena of different objects with the same name and the same object with different names often appeared to Floccularia luteovirens produced in different areas, so morphological identification was difficult to realize effective distinguishment. More difficultly, it is impossible to screen out high-quality strains with high contents of total soluble proteins, total soluble amino acids, total polyphenols, total polysaccharides and total fat, and strong antioxidant activity by the morphological method. A DNA barcode molecular identification technology is a molecular biology technology based on DNA barcodes (conserved and stable genetic DNA sequences in a genome) to recognize and identify species and excellent quality. It is the effective supplement and expansion of traditional breeding methods, and can accurately and effectively identify samples when samples are incomplete in morphologies or lack morphological structures (processed products such as powder, etc.).

In the existing DNA barcode technology, non-coding regions or conserved gene sequences in ITS (internal transcribed spacer in ribosomal RNA) and mitochondria are mainly used for species object identification; the operation of restriction fragment length polymorphism (RFLP) is very complicated; reliability and repeatability of results are poor; random amplified polymorphic DNA (RAPD) is easily disturbed, which requires a high technical level of operators and is difficult to popularize in assisted breeding; and single nucleotide polymorphism (SNP) has high requirements for equipment and high cost.

Therefore, in view of shortcomings of traditional breeding methods that the breeding of Floccularia luteovirens strains is not accurate enough, time-consuming and labor-consuming, an urgent problem to be solved by those skilled in the art is how to provide a DNA barcode that can accurately and quickly identify a strain of Floccularia luteovirens and realize high-quality breeding, which has the characteristics of low cost, high efficiency, simple operation, stable and reliable results and good repeatability.

### SUMMARY

In view of this, the present invention provides a DNA barcode for screening Floccularia luteovirens with high total fat content.

In order to achieve the above purpose, the present invention adopts the following technical solution:

A DNA barcode for screening a total fatty acid content index of Floccularia luteovirens, wherein a nucleotide sequence of the DNA barcode includes one or more of:

SEQ ID NO:4,
and/or a combination of SEQ ID NO:3 and SEQ ID NO:4,
and/or a combination of SEQ ID NO:7 and SEQ ID NO:8,
and/or a combination of SEQ ID NO:9,
and/or SEQ ID NO:12,
and/or a combination of SEQ ID NO:12 and SEQ ID NO:14,
and/or a combination of SEQ ID NO:12, SEQ ID NO:13 and SEQ ID NO:14,
and/or SEQ ID NO:18,
and/or a combination of SEQ ID NO:17 and SEQ ID NO:18.

According to the present invention, based on all simple sequence repeats (SSR) in the whole genome of Floccularia luteovirens, fluorescent PCR amplification is carried out; a DNA barcode which effectively corresponds to total fatty acid content is established; through comparison between the amplified fragments and the DNA barcode of the present invention, quick and accurate screening of Floccularia luteovirens strains with high total fatty acid content can be realized; and beneficial assistance can be provided for breeding of Floccularia luteovirens.

Another purpose of the present invention is to provide a primer group for amplifying the DNA barcode for screening the total fatty acid content index of Floccularia luteovirens, wherein a nucleotide sequence of the primer group includes one or more groups of:
SEQ ID NO:1 and SEQ ID NO:2,
and/or SEQ ID NO:5 and SEQ ID NO:6,
and/or SEQ ID NO:10 and SEQ ID NO: 11,
and/or SEQ ID NO:15 and SEQ ID NO:16.

As a preferred technical solution of the present invention, the nucleotide sequence of the primer group includes: such as SEQ ID NO:1 and SEQ ID NO:2, SEQ ID NO: 5 and SEQ ID NO:6, SEQ ID NO:10 and SEQ ID NO:11, and SEQ ID NO:15 and SEQ ID NO:16.

Different primer groups of the present invention can be used alone or in combination to screen the total fatty acid content of Floccularia luteovirens; and when all primer groups are used together, the screening accuracy is the highest.

Another purpose of the present invention is to provide a method for screening Floccularia luteovirens with a total fatty acid content index, which comprises the following steps:
S1, extracting genomic DNA of a sample to be tested;
S2, taking the genomic DNA in S1 as a template, and performing a fluorescent PCR amplification reaction on one or more groups of the above primers to obtain an amplification product;
S3, detecting the amplification product of S2 by capillary fluorescence electrophoresis, and judging by the number of fragments, the number of SSR loci, SSR repetitive elements and repeating times of the amplification product.

As a preferred technical solution of the present invention, a judgment standard in step S3 is:
when the primer group of SEQ ID NO:1 and SEQ ID NO:2 is amplified to obtain a 271 bp fragment containing 8 TGA repetitive elements only;
and/or the primer group of SEQ ID NO:5 and SEQ ID NO:6 is amplified to obtain a 241 bp fragment containing 10 CAG repetitive elements;
and/or the primer group of SEQ ID NO:10 and SEQ ID NO:11 is amplified to obtain a 236 bp fragment containing 6 AT repetitive elements only;
and/or the primer group of SEQ ID NO:15 and SEQ ID NO:16 is amplified to obtain a 270 bp fragment containing 10 TAA repetitive elements, the Floccularia luteovirens is judged as Floccularia luteovirens with high total fatty acid content.

As a preferred technical solution of the present invention, a reaction system of the fluorescent PCR amplification reaction in step S2 is:

5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

Further preferably, the concentrations of the forward primer, the reverse primer and the M13 primer with fluorescence are all 10 uM.

As a preferred technical solution of the present invention, a fluorescent PCR amplification reaction procedure in step S2 is:

performing pre-denaturation at 95° C. for 3 min, denaturation at 95° C. for 30 s, PCR annealing during the decrease from 62° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.

Further another purpose of the present invention is to provide application of the DNA barcode and/or the primer group in preparation of a product for screening Floccularia luteovirens with a total fatty acid content index.

Further another purpose of the present invention is to provide a product for screening Floccularia luteovirens with a total fatty acid content index, which comprises one or more of the above primer groups and satisfies one or more of the following standards:
the primer group of SEQ ID NO:1 and SEQ ID NO:2 is amplified to obtain a 271 bp fragment containing 8 TGA repetitive elements only;
and/or the primer group of SEQ ID NO:5 and SEQ ID NO:6 is amplified to obtain a 241 bp fragment containing 10 CAG repetitive elements;
and/or the primer group of SEQ ID NO:10 and SEQ ID NO:11 is amplified to obtain a 236 bp fragment containing 6 AT repetitive elements only;

and/or the primer group of SEQ ID NO:15 and SEQ ID NO:16 is amplified to obtain a 270 bp fragment containing 10 TAA repetitive elements.

As a preferred technical solution of the present invention, the product is a kit.

According to the above technical solution, compared with the prior art, the present invention discloses and provides the DNA barcode for screening Floccularia luteovirens with high total fat content. Compared with the prior art, the present invention can accurately and quickly identify Floccularia luteovirens strains and realize a DNA barcode technology of high-quality breeding. The present invention is characterized by low cost, high efficiency, simple operation, stable and reliable results and good repeatability.

Compared with a traditional breeding method and other existing DNA barcode technologies, the present invention has the advantages of time saving, labor saving, money saving, accuracy and high efficiency, plays a positive role in original place identification and genetic breeding of high-quality Floccularia luteovirens, and also provides an effective method for identification and protection of germplasm resources.

### DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a comparison result diagram of total fat contents in embodiment, reference example 1 and reference example 2 according to the present invention, wherein reference example 1, reference example 2 and embodiment are listed in order from left to right;

FIG. 2A is a diagram of PCR amplification using primer 1 in reference example 1 according to the present invention;

FIG. 2B is a diagram of PCR amplification using primer 1 in reference example 2 according to the present invention;

FIG. 2C is a diagram of PCR amplification using primer 1 in one embodiment according to the present invention;

FIG. 3A is a diagram of PCR amplification using primer 2 in reference example 1 according to the present invention;

FIG. 3B is a diagram of PCR amplification using primer 2 in reference example 2 according to the present invention;

FIG. 3C is a diagram of PCR amplification using primer 2 in one embodiment according to the present invention;

FIG. 4A is a diagram of PCR amplification using primer 3 in reference example 1 according to the present invention;

FIG. 4B is a diagram of PCR amplification using primer 3 in reference example 2 according to the present invention;

FIG. 4C is a diagram of PCR amplification using primer 3 in one embodiment according to the present invention;

FIG. 5A is a diagram of PCR amplification using primer 4 in reference example 1 according to the present invention;

FIG. 5B is a diagram of PCR amplification using primer 4 in reference example 2 according to the present invention; and

FIG. 5C is a diagram of PCR amplification using primer 4 in one embodiment according to the present invention.

### DETAILED DESCRIPTION

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Embodiments of the present invention disclose a DNA barcode for screening Floccularia luteovirens with high total fat content.

### Embodiment 1

Establishment of a DNA Barcode of Floccularia luteovirens

Samples of Floccularia luteovirens are collected from Dangxiong County of Tibet Autonomous Region, Qilian County of Qinghai Province and Shiqu County of Sichuan Province and are treated by genome sequencing; and SSR loci in the genome sequences are analyzed by MISA program.

Primers are designed for PCR amplification of the SSR loci; the primers that can amplify the corresponding fragments are reserved; and invalid primers are discarded.

Samples of Floccularia luteovirens from Dangxiong County of Tibet Autonomous Region, Qilian County of Qinghai Province and Shiqu County of Sichuan Province are selected for testing of total fatty acid content.

The samples from the above three original places are amplified by effective primers and detected by capillary electrophoresis. The simple sequence repeat (SSR) locus corresponding to a total fatty acid content is established by analysis. Finally, four pairs of primers (see Table 1) are obtained. The four pairs of primers are used to amplify the sample genome; and the fragment polymorphism obtained can assist in screening Floccularia luteovirens with high total fatty acid content.

TABLE 1
Specific primers for screening of Floccularia
luteovirens strains with high
total fat content

| | | Denaturation | | Denaturation | |
|---|---|---|---|---|---|
| | | temperature | Reverse | temperature | SSR |
| No. | Forward primer | (° C.) | primer | (° C.) | element |
| 1 | TGTAAAACGACGGC | 60.039 | AGGTATAGG | 60.034 | TGA |
| | CAGTAGTGGTTCTCG | | AGGCGGGTG | | |
| | CTTACACCG, | | TT, | | |
| | as shown | | as shown in | | |
| | in SEQ ID No: 1 | | SEQ ID No: 2 | | |
| 2 | TGTAAAACGACGGC | 60.036 | GGCTCCTAGC | 59.968 | ACA |
| | CAGTACAGGGCTCAT | | GCTATTCGTT, | | |
| | TGTACCGTG, | | as shown in | | |
| | as shown in | | SEQ ID No: 6 | | |
| | SEQ ID No: 5 | | | | |
| 3 | TGTAAAACGACGGC | 59.966 | TGCCTGGTTG | 59.599 | GT |
| | CAGTACAACACATG | | ATGAAGCTG | | |
| | CACCAATGGC, as | | A, | | |
| | shown in | | as shown in | | |
| | SEQ ID No: 10 | | SEQ ID No: 11 | | |
| 4 | TGTAAAACGACGGC | 60.038 | ATCTCCGTGA | 60.25 | TAA |
| | CAGTGCCCGTTTGAT | | TCATGCCTGC, | | |
| | GCAACATGT, as | | as shown in | | |
| | shown in | | SEQ ID No: 16 | | |
| | SEQ ID No: 15 | | | | |

### Embodiment 2

SSR specific primer amplification of Floccularia luteovirens strains with high total fatty acid content

(1) Screening and verification of total fat index

Samples from Dangxiong County of Tibet Autonomous Region are taken as test example of the present invention. Freeze-dried powder of fruiting body samples is sieved by a 50-mesh sieve; 100 mL of chloroform and MTBE solution is added into 10 g of dry powder; extraction lasts for 30 min with assistance of 300 W ultrasonic waves; centrifugation is carried out for 30 min at 5000 r/min; the sample is removed through filtration; and through spin-drying at 60° C., an extracting solvent is used to prepare a total fat extracting solution.

Reference example 1: samples from Qilian County of Qinghai Province (treatment method is the same as above).

Reference example 2: samples from Shiqu County of Sichuan Province (treatment method is the same as above).

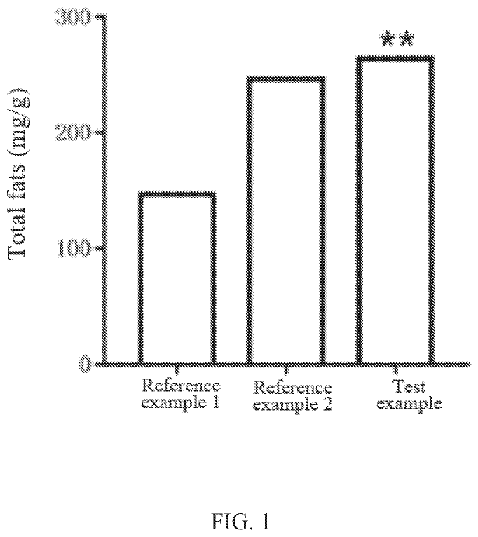

A total fat content of the extract is detected by a Gas Chromatography-Mass Spectrometer after saponification and methyl esterification. Please refer to Suolang Yangzong et al. (Tibet of Science and Technology, 2019, 5:16-19) for details. The result is converted to mg/g. The total fatty acid content in Floccularia luteovirens from Qilian County of Qinghai Province is 146.65(±0.64) mg/g, which is determined as reference example 1; the total fatty acid content in Floccularia luteovirens from Shiqu County of Sichuan Province is 245.95(±0.21) mg/g, which is determined as reference example 2; and the total fatty acid content in Floccularia luteovirens from Dangxiong County of Tibet Autonomous Region is 264.05(+0.21) mg/g, which is determined as test example (see FIG. 1).

(2) The genome of Floccularia luteovirens samples is extracted by an Ezup column fungus genomic DNA extraction kit (No. B518259) of Sangon Biotech (Shanghai) Co., Ltd., and diluted to 20 ng/μL for fluorescent PCR amplification.

(3) Fluorescent PCR amplification of the SSR DNA barcode is carried out with primers in Table 1.

Fluorescent PCR amplification reaction system (10 μL): 5 μL of 2×Taq PCR MasterMix, 1 μL of template (genomic DNA), 0.1 μL of forward primer, 0.4 μL of reverse primer (concentrations of the forward primer and the reverse primer are both 10 uM), and 0.4 μL of M13 primer with fluorescence (concentration of 10 uM), wherein a volume is fixed to 10 μL with sterile deionized water.

Reaction conditions: performing pre-denaturation at 95° C. for 3 min, denaturation at 95° C. for 30 s, PCR annealing during the decrease from 62° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.

(4) After quantitative dilution of the PCR product, 1 μL of a PCR diluted products is denatured with 9 μL of formamide (including 1% internal standard), and then subjected to capillary fluorescence electrophoresis detection by a DNA sequencer ABI 3730x1. The internal standard, which is a molecular weight internal standard (also known as internal lane standard) LIZ-500 bp, is composed of 16 double-stranded DNA fragments labeled with LIZ fluorescein (orange), with molecular weights of 35, 50, 75, 100, 139, 150, 160, 200, 250, 300, 340, 350, 400, 450, 490 and 500 bp respectively. The fragment size in the amplification result electropherogram is equal to the actual bp number of the amplified fragment plus the M13 fluorescent primer about 18 bp (with error of 1-2 bp). The amplified capillary electrophoresis peak is combined with the sequencing result; and the peak number indicates the number of heterozygous amplified fragments of the gene.

(5) The above methods are used to identify Floccularia luteovirens in test example, reference example 1 and reference example 2.

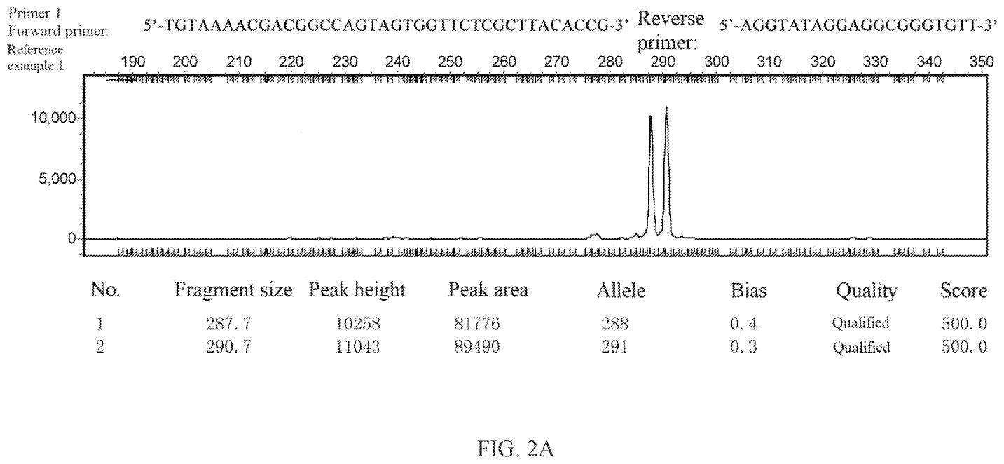

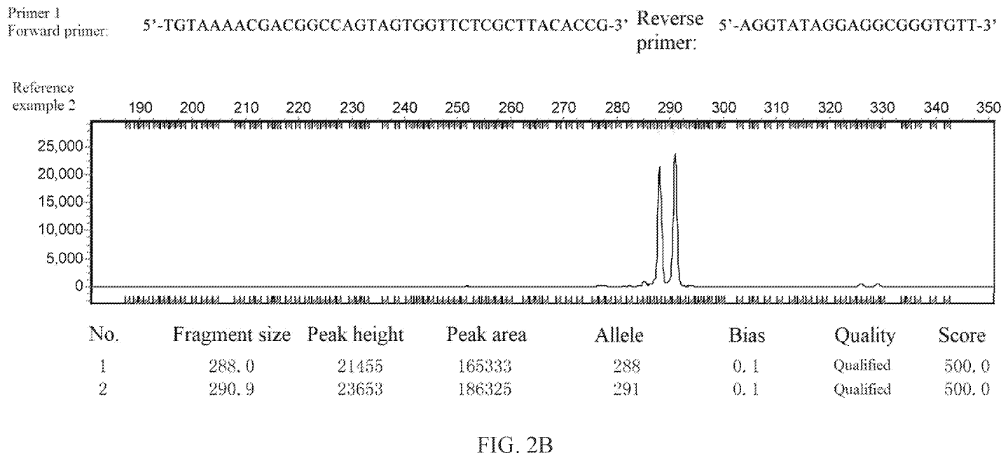

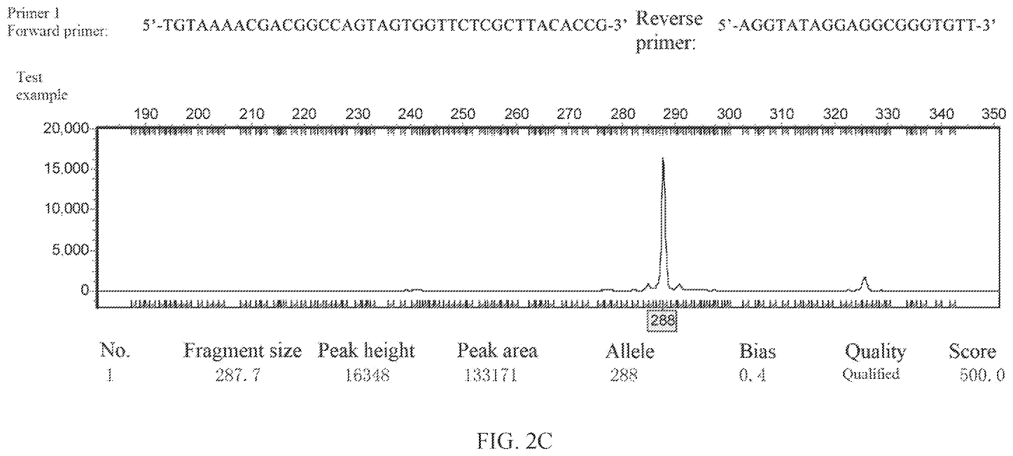

Amplification results of primer 1 are shown in FIG. 2A-2C When primer 1 is used for fluorescent PCR amplification, two fragments (two peaks) are obtained through amplification, which contain two SSR loci; and the SSR repetitive element is TGA. The amplified fragment obtained in the test example is characterized by containing a 271 bp fragment with 8 repeats of TGA.

Amplified fragments of primer 1: (the statistical fragment length of electropherogram includes the M13 fluorescent primer; the specific sequence shows that the M13 fluorescent primer sequence (17 bp) with error of 1 bp is removed; and the underlined part is an SSR repetitive element.)

271 bp amplified fragment sequence:
AGTGGTTCTCGCTTACACCGAACCTGATTCTGAATACTCTGCAGA
CGCAGATGAAGAAATGACACCTGAGGCGATTATCCACGCTCGTCA
GGTTGTTGGGCAACAAGGGCGTCTTGGTTACTTTGACCTACATCC
AGAACGAAGACCTGCCGCTGTCAATCAAAATGATGAAGACCCTAG
ATTGCGCCTTGCCAGAGACGACAGTGATGATGATGATGATGATGA
TGAACAAGTATATGGTCGCTCCGAGCAACACCCGCCTCCTATACC
T
(as shown in SEQ ID NO: 3);
274 bp amplified fragment sequence:
AGTGGTTCTCGCTTACACCGAACCTGATTCTGAATACTCTGCAGA
CGCAGATGAAGAAATGACACCTGAGGCGATTATCCACGCTCGTCA
GGTTGTTGGGCAACAAGGGCGTCTTGGTTACTTTGACCTACATCC
AGAACGAAGACCTGCCGCTGTCAATCAAAATGATGAAGACCCTAG
ATTGCGCCTTGCCAGAGACGACAGTGATGATGATGATGATGATGA
TGATGAACAAGTATATGGTCGCTCCGAGCAACACCCGCCTCCTAT
ACCT
(as shown in SEQ ID NO: 4);

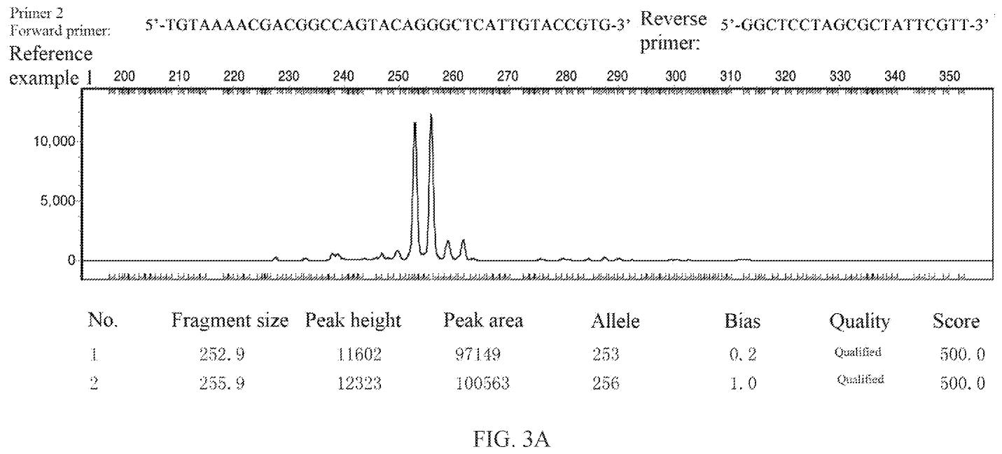

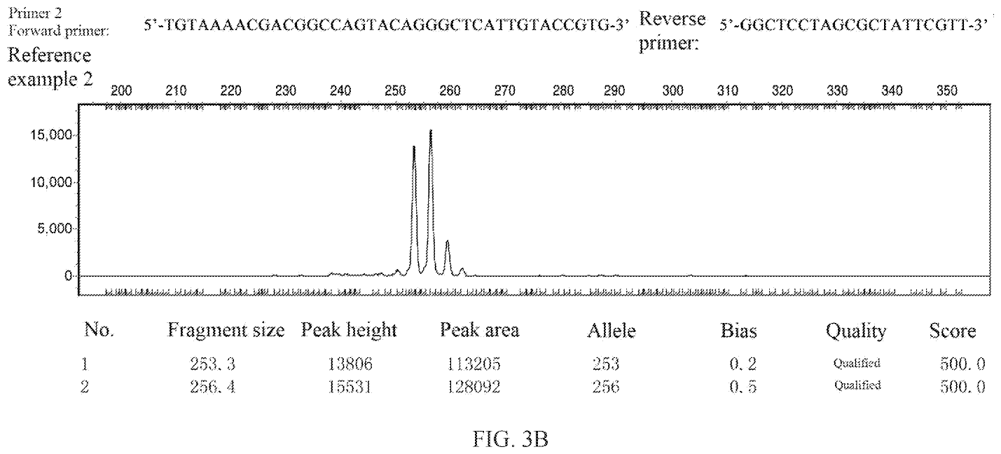

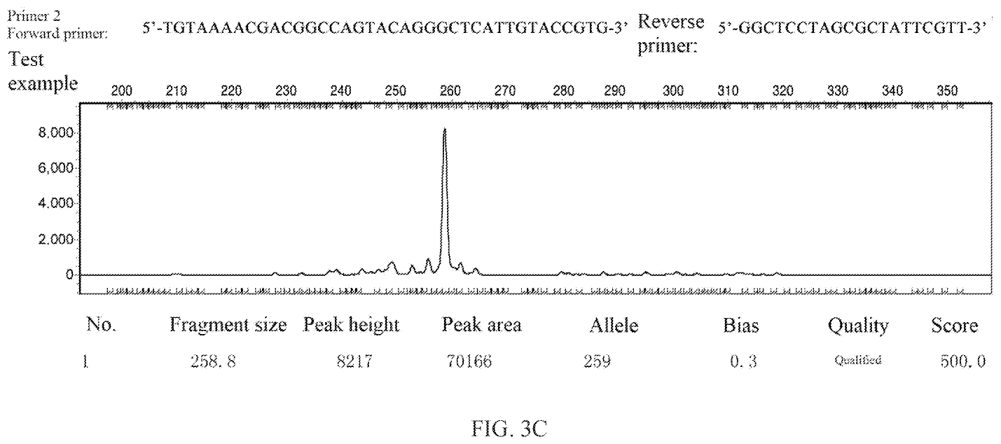

Amplification results of primer 2 are shown in FIGS. 3A-3C. When primer 2 is used for fluorescent PCR amplification, three fragments (three peaks) are obtained, which contain three SSR loci; and the SSR repetitive element is ACA. The amplified fragment obtained in the test example is characterized by containing a 241 bp fragment with 10 repeats of CAG.

Amplified fragments of primer 2: (the statistical fragment length of electropherogram includes the M13 fluorescent primer; the specific sequence shows that the M13 fluorescent primer sequence (18 bp) is removed; and the underlined part is an SSR repetitive element.)

235 bp amplified fragment sequence:
ACAGGGCTCATTGTACCGTGCTCAGACGGGCACCCTATTCAAACG
CATTTGGAGAACACCCGAAATGGCGATGCTGTTTGGTGGGGATGC
GGTGAGCTTTGCATGGGTTTGCAGGAAGAGGATGAGTCGGATTTA
GAGGATGCAGATGAACGGACTTTGCTACAACAACAACAACAACAA
CAACATGGGCATGCTTCATCACCATTGCCCAATCGAACGAATAGC
GCTAGGAGCC
(as shown in SEQ ID NO: 7);
238 bp amplified fragment sequence;
ACAGGGCTCATTGTACCGTGCTCAGACGGGCACCCTATTCAAACG
CATTTGGAGAACACCCGAAATGGCGATGCTGTTTGGTGGGGATGC
GGTGAGCTTTGCATGGGTTTGCAGGAAGAGGATGAGTCGGATTTA
GAGGATGCAGATGAACGGACTTTGCTACAACAACAACAACAACAA
CAACAACATGGGCATGCTTCATCACCATTGCCCAATCGAACGAAT
AGCGCTAGGAGCC
(as shown in SEQ ID NO: 8);
241 bp amplified fragment sequence:
ACAGGGCTCATTGTACCGTGCTCAGACGGGCACCCTATTCAAACG
CATTTGGAGAACACCCGAAATGGCGATGCTGTTTGGTGGGGATGC
GGTGAGCTTTGCATGGGTTTGCAGGAAGAGGATGAGTCGGATTTA
GAGGATGCAGATGAACGGACTTTGCTACAACAACAACAACAACAA
CAACAACAACATGGGCATGCTTCATCACCATTGCCCAATCGAACG
AATAGCGCTAGGAGCC
(as shown in SEQ ID NO: 9).

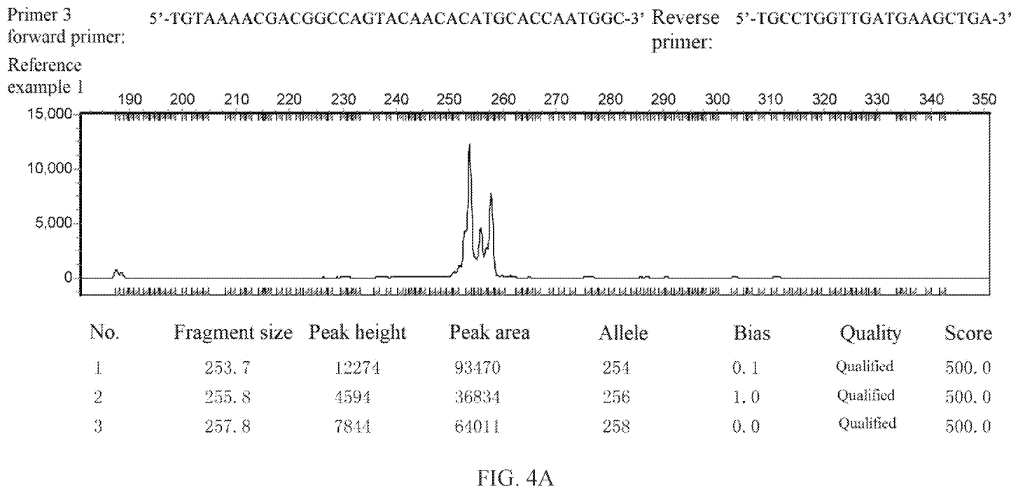

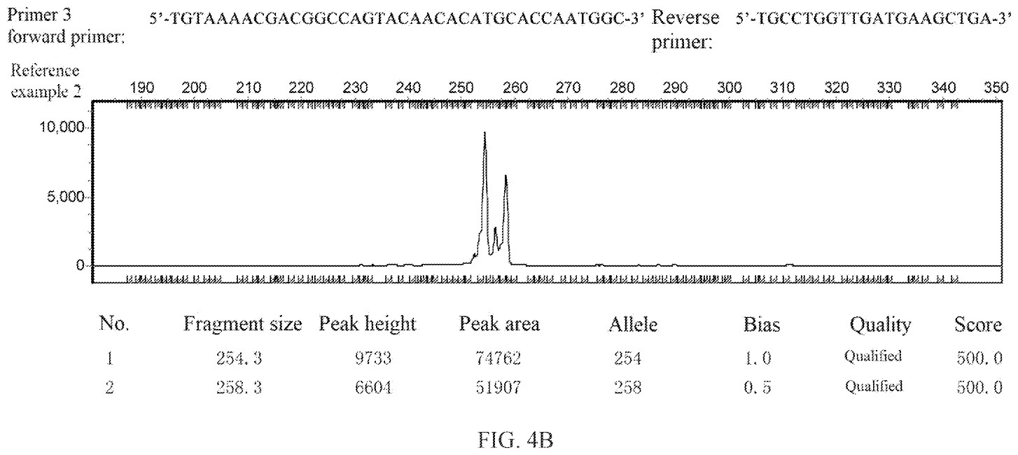

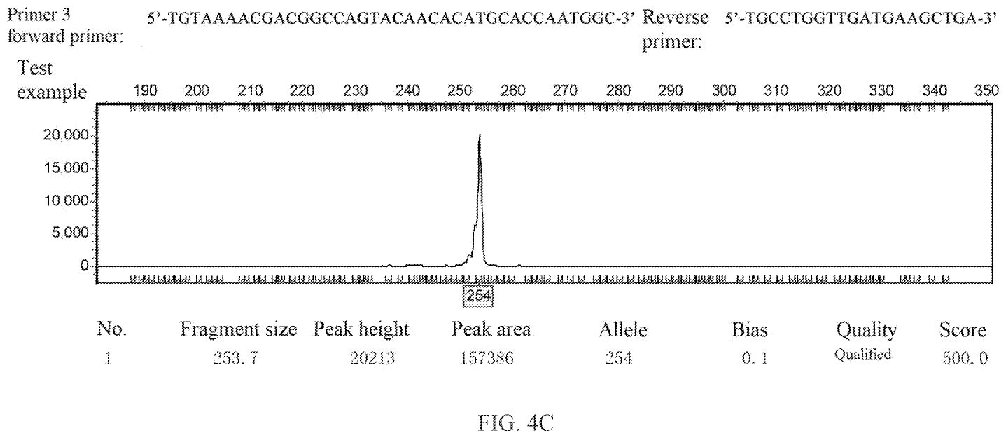

Amplification results of primer 3 are shown in FIGS. 4A-4C When primer 3 is used for fluorescent PCR amplification, three fragments (three peaks) are obtained, which contain three SSR loci; and the SSR repetitive element is GT. The amplified fragment obtained in the test example is characterized by containing a 236 bp fragment with 6 repeats.

Amplified fragments of primer 3: (the statistical fragment length of electropherogram includes the M13 fluorescent primer; the specific sequence shows that the M13 fluorescent primer sequence (18 bp) is removed; and the underlined part is an SSR repetitive element.)

236 bp amplified fragment sequence:
ACAACACATGCACCAATGGCCATCAAATAATAATAAAATAACTAT
TCATTCAGCCTTAGGGAACAGCGTACGAGTCGGACATTTACAATA
GTGTGTGTGTGTACTGCATTCAACCAAACTTTGGACTGTAACGTG
TACTTCACTCTGCCAACATGATCATGAGGTGAAGCATGTTTTCAA
GTCACTCACCACACTCTCTCACACACATGATGGCTTTCAGCTTCA
TCAACCAGGCA
(as shown in SEQ ID NO: 12);
238 bp amplified fragment sequence:
ACAACACATGCACCAATGGCCATCAAATAATAATAAAATAACTAT
TCATTCAGCCTTAGGGAACAGCGTACGAGTCGGACATTTACAATA
GTGTGTGTGTGTGTACTGCATTCAACCAAACTTTGGACTGTAACG
TGTACTTCACTCTGCCAACATGATCATGAGGTGAAGCATGTTTT
CAAGTCACTCACCACACTCTCTCACACACATGATGGCTTTCAGC
TTCATCAACCAGGCA
(as shown in SEQ ID NO: 13);
240 bp amplified fragment sequence:
ACAACACATGCACCAATGGCCATCAAATAATAATAAAATAACTAT
TCATTCAGCCTTAGGGAACAGCGTACGAGTCGGACATTTACAATA
GTGTGTGTGTGTGTGTACTGCATTCAACCAAACTTTGGACTGTAA
CGTGTACTTCACTCTGCCAACATGATCATGAGGTGAAGCATGTTT
TCAAGTCACTCACCACACTCTCTCACACACATGATGGCTTTCAGC
TTCATCAACCAGGCA
(as shown in SEQ ID NO: 14).

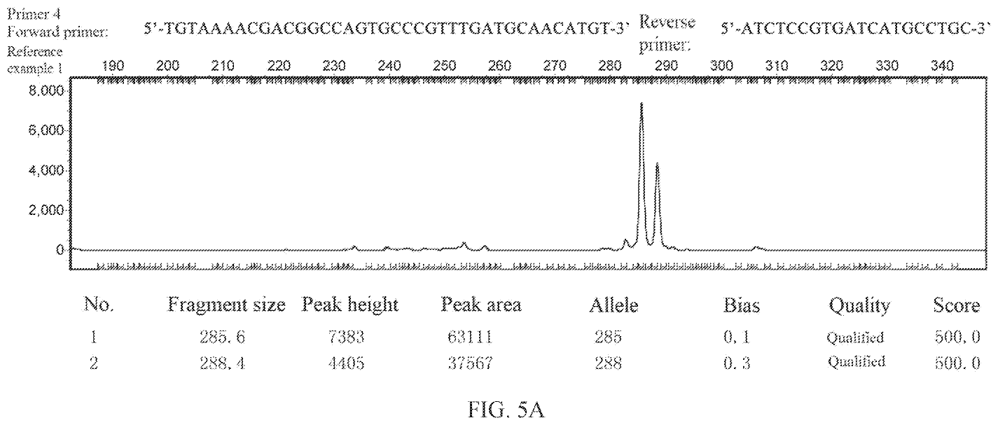

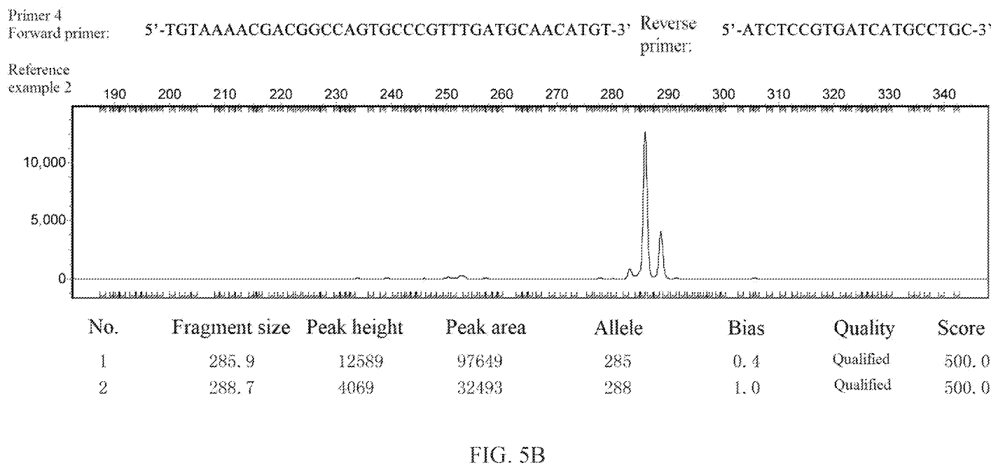

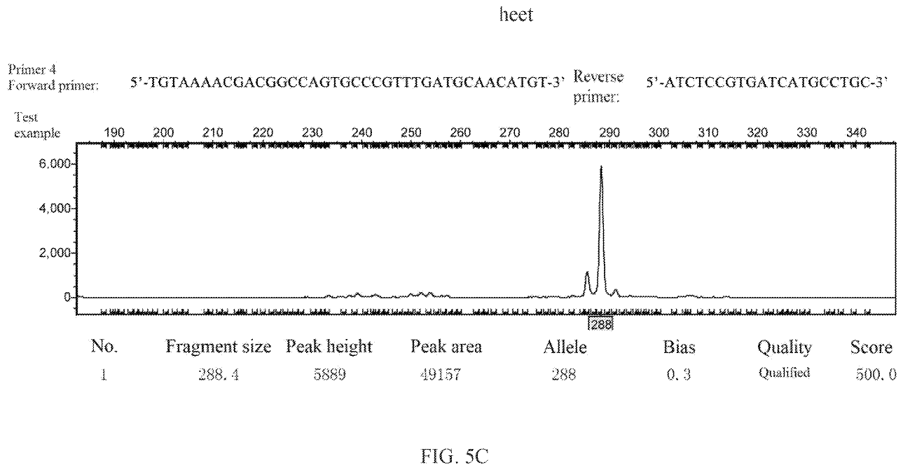

Amplification results of primer 4 are shown in FIGS. 5A-5C. When primer 4 is used for fluorescent PCR amplification, two fragments (two peaks) are obtained, which contain two SSR loci; and the SSR repetitive element is TAA. The amplified fragment obtained in the test example is characterized by containing a 270 bp fragment with 10 repeats.

Amplified fragments of primer 4: (the statistical fragment length of electropherogram includes the M13 fluorescent primer; the specific sequence shows that the M13 fluorescent primer sequence (18 bp) is removed; and the underlined part is an SSR repetitive element.)

267 bp amplified fragment sequence;
GCCCGTTTGATGCAACATGTTGATTGACGACTAGATTGGACCTGC
AATGCAAACTAATAATAATAATAATAATAATAATAAAGTCAGCCG
GCGTGTAGAAATCTGACACTTACAGTAAGAGTGCAGATCACCCAA
GTAGGTTATGAGAAGTATATATATACACTTTATACGATTACGTGT
AACATCACATGATATCACTTTTTACTTTCTACGTGACATTTCCCA
CTCCAAAGTCCAATACTTCCTTGCAGGCATGATCACGGAGAT
(as shown in SEQ ID NO: 17);
270 bp amplified fragment sequence:
GCCCGTTTGATGCAACATGTTGATTGACGACTAGATTGGACCTGC
AATGCAAACTAATAATAATAATAATAATAATAATAATAAAGTCAG
CCGGCGTGTAGAAATCTGACACTTACAGTAAGAGTGCAGATCACC
CAAGTAGGTTATGAGAAGTATATATATACACTTTATACGATTACG
TGTAACATCACATGATATCACTTTTTACTTTCTACGTGACATTTC
CCACTCCAAAGTCCAATACTTCCTTGCAGGCATGATCACGGAGAT
(as shown in SEQ ID NO: 18).

Through comprehensive analysis of maps and sequencing results of test example, reference example 1 and reference example 2, the characteristic information of the DNA barcode of Floccularia luteovirens with high total fatty acid content is shown in Table 2.

TABLE 2
DNA barcode characteristics of Floccularia luteovirens
with high total fatty acid content

| | Primer 1 | Primer 2 | Primer 3 | Primer 4 |
|---|---|---|---|---|
| Total | amplified | amplified | amplified | amplified |
| fatty acid | fragment | fragment | fragment | fragment |
| content | (SSR) | (SSR) | (SSR) | (SSR) |
| Reference | 271 bp (8TGA) | 235 bp (8CAG) | 236 bp (6GT) | 267 bp |
| example 1 | 274 bp (9TGA) | 238 bp (9CAG) | 238 bp (7GT) | (9TAA) |
| Low content | | | 240 bp (8GT) | 270 bp |
| | | | | (10TAA) |
| Reference | 271 bp (8TGA) | 235 bp (8CAG) | 236 bp (6GT) | 267 bp |
| example 2 | 274 bp (9TGA) | 238 bp (9CAG) | 240 bp (8GT) | (9TAA) |
| Low content | | | | 270 bp |
| | | | | (10TAA) |
| Test example | 271 bp (8TGA) | 241 bp (10CAG) | 236 bp (6GT) | 270 bp |
| High content | | | | (10TAA) |

Primer 1 carries out amplification to obtain the 271 bp fragment containing 8 TGA repetitive elements (as shown in SEQ ID NO:3); primer 2 carries out amplification to obtain the 241 bp fragment containing 10 CAG repetitive elements (as shown in SEQ ID NO:9); primer 3 carries out amplification to obtain the 236 bp fragment containing 6 GT repetitive elements (as shown in SEQ ID NO:12); and primer 4 carries out amplification to obtain the 270 bp fragment containing 10 TAA repetitive elements (as shown in SEQ ID NO:18). When primers 1, 2, 3 and 4 are used for comprehensive detection and judgment respectively, the accuracy of screening the total fatty acid content index of Floccularia luteovirens is best.

### Embodiment 3

Screening and Verification of Total Fatty Acid Content Index of Floccularia luteovirens

A DNA barcode of a total fatty acid content of Floccularia luteovirens is verified by blind testing.

Step 1, blind testing: taking samples from Dangxiong County of Tibet Autonomous Region with the total fatty acid content higher than or equal to 264.05 mg/g as test example; taking samples from Qilian County of Qinghai Province and Shiqu County of Sichuan Province with the total fat content lower than 264.05 mg/g (significance p<0.05) as reference group 1 and reference group 2; and taking 16 samples respectively for blind testing;

step 2, testing: using primers (SEQ ID NO:1 and SEQ ID NO:2, SEQ ID NO:5 and SEQ ID NO:6, SEQ ID NO:10 and SEQ ID NO:11, and SEQ ID NO:15 and SEQ ID NO:16) for amplification and capillary electrophoresis. One or more pairs of primer groups can be combined for amplification; and DNA barcode characteristics can be used to distinguish the blind testing samples;

step 3, unblinding: results are shown in Table 3. The unblinding results of 48 samples for distinguishing a total fatty acid content with the total fatty acid content barcode characteristics are all correct, indicating that the DNA barcode of the total fatty acid content is suitable for screening the total fatty acid content character.

TABLE 3
Screening results of amplification using a pair of primers

| | Primer 1 | Primer 2 | Primer 3 | Primer 4 | | |
|---|---|---|---|---|---|---|
| Blind | amplified | amplified | amplified | amplified | Unblinding | Distinguishment |
| testing No. | fragment (SSR) | fragment (SSR) | fragment (SSR) | fragment (SSR) | No. | results |
| 3, 10, 11, 18, | 271 bp (8TGA) | 235 bp (8CAG) | 236 bp (6GT) | 267 bp (9TAA) | 1-16 | Low content in |
| 21, 23, 27, 29, | 274 bp (9TGA) | 238 bp (9CAG) | 238 bp (7GT) | 270 bp (10TAA) | | reference example 1 |
| 31, 32, 36, 37, | | | 240 bp (8GT) | | | |
| 40, 41, 44, 48 | | | | | | |
| 1, 6, 9, 13, 16, | 271 bp (8TGA) | 235 bp (8CAG) | 236 bp (6GT) | 267 bp (9TAA) | 17-32 | Low content in |
| 17, 20, 24, 28, | 274 bp (9TGA) | 238 bp (9CAG) | 240 bp (8GT) | 270 bp (10TAA) | | reference example 2 |
| 30, 33, 38, 39, | | | | | | |
| 42, 46, 47 | | | | | | |
| 2, 4, 5, 7, 8, 12, | 271 bp (8TGA) | 241 bp (10CAG) | 236 bp (6GT) | 270 bp (10TAA) | 33-48 | High content in |
| 14, 15, 19, 22, 25, | | | | | | test example |
| 26, 34, 35, 43, 45 | | | | | | |

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A DNA barcode for screening a total fatty acid content index of Floccularia luteovirens, wherein a nucleotide sequence of the DNA barcode comprises one or more of:
SEQ ID NO:4,
and/or a combination of SEQ ID NO:3 and SEQ ID NO:4,
and/or a combination of SEQ ID NO:7 and SEQ ID NO:8,
and/or a combination of SEQ ID NO:9,
and/or SEQ ID NO:12,
and/or a combination of SEQ ID NO: 12 and SEQ ID NO:14,
and/or a combination of SEQ ID NO: 12, SEQ ID NO:13 and SEQ ID NO:14,
and/or SEQ ID NO:18,
and/or a combination of SEQ ID NO:17 and SEQ ID NO:18.

2. A primer group for amplifying the DNA barcode for screening the total fatty acid content index of Floccularia luteovirens according to claim 1, wherein a nucleotide sequence of the primer group comprises one or more groups of:
SEQ ID NO: 1 and SEQ ID NO:2,
and/or SEQ ID NO:5 and SEQ ID NO:6,
and/or SEQ ID NO:10 and SEQ ID NO:11,
and/or SEQ ID NO:15 and SEQ ID NO:16.

3. The primer group according to claim 2, wherein the nucleotide sequence of the primer group comprises: such as SEQ ID NO:1 and SEQ ID NO:2, SEQ ID NO: 5 and SEQ ID NO:6, SEQ ID NO:10 and SEQ ID NO:11, and SEQ ID NO:15 and SEQ ID NO:16.

4. A method for screening Floccularia luteovirens with a total fatty acid content index, comprising the following steps:
S1, extracting genomic DNA of a sample to be tested;
S2, taking the genomic DNA in S1 as a template, and performing a fluorescent PCR amplification reaction on one or more of the primer groups according to claim 2 to obtain an amplification product;
S3, detecting the amplification product of S2 by capillary fluorescence electrophoresis, and judging by the number of fragments, the number of SSR loci, SSR repetitive elements and repeating times of the amplification product.

5. The method for screening Floccularia luteovirens with the total fatty acid content index according to claim 4, wherein a judgment standard in step S3 is:
when the primer group of SEQ ID NO:1 and SEQ ID NO:2 is amplified to obtain a 271 bp fragment containing 8 TGA repetitive elements only;
and/or the primer group of SEQ ID NO:5 and SEQ ID NO:6 is amplified to obtain a 241 bp fragment containing 10 CAG repetitive elements;
and/or the primer group of SEQ ID NO:10 and SEQ ID NO:11 is amplified to obtain a 236 bp fragment containing 6 AT repetitive elements only;
and/or the primer group of SEQ ID NO:15 and SEQ ID NO:16 is amplified to obtain a 270 bp fragment containing 10 TAA repetitive elements, the Floccularia luteovirens is judged as Floccularia luteovirens with high total fatty acid content.

6. The method for screening Floccularia luteovirens with the total fatty acid content index according to claim 4, wherein a reaction system of the fluorescent PCR amplification reaction in step S2 is:
5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

7. The method for screening Floccularia luteovirens with the total fatty acid content index according to claim 6, wherein the concentrations of the forward primer, the reverse primer and the M13 primer with fluorescence are all 10 uM.

8. The method for screening Floccularia luteovirens with the total fatty acid content index according to claim 4, wherein a fluorescent PCR amplification reaction procedure in step S2 is:
performing pre-denaturation at 95° C. for 3 min, denaturation at 95° C. for 30 s, PCR annealing during the decrease from 62° C. to 55ºC for 30 s, and extension at 72° C. for 30 s, with a total of 10 cycles; performing denaturation at 95° C. for 30 s, annealing at 52° C. for 30 s, and extension at 72ºC for 30 s, with a total of 25 cycles; performing final extension at 72ºC for 20 min; and after heat preservation at 4ºC for 6 h, using the product for fluorescence capillary electrophoresis detection.

9. An application of the DNA barcode according to claim 1 in preparation of a product for screening Floccularia luteovirens with a total fatty acid content index.

10. An application of the primer group according to claim 2 in preparation of a product for screening Floccularia luteovirens with a total fatty acid content index.

11. A product for screening high-quality Floccularia luteovirens with a total fatty acid content index, comprising one or more of the primer groups according to claim 2 and satisfying one or more of the following standards:
the primer group of SEQ ID NO:1 and SEQ ID NO:2 is amplified to obtain a 271 bp fragment containing 8 TGA repetitive elements only;
and/or the primer group of SEQ ID NO:5 and SEQ ID NO:6 is amplified to obtain a 241 bp fragment containing 10 CAG repetitive elements;
and/or the primer group of SEQ ID NO:10 and SEQ ID NO:11 is amplified to obtain a 236 bp fragment containing 6 AT repetitive elements only;
and/or the primer group of SEQ ID NO:15 and SEQ ID NO:16 is amplified to obtain a 270 bp fragment containing 10 TAA repetitive elements.
